(19) United States
(12) Patent Application Publication
(10) Pub. No.: US 2024/0382455 A1
(43) Pub. Date: Nov. 21, 2024

(54) COMPOSITION CONTAINING 1H-PYRAZOLE-3-AMIDE-BASED COMPOUND DERIVATIVE FOR PREVENTION OR TREATMENT OF FIBROSIS

(71) Applicant: TSD LIFE SCIENCES CO., LTD., Seoul (KR)

(72) Inventors: Wook KIM, Suwon-si (KR); Sung-Hwa YOON, Suwon-si (KR); Ji Hye HAN, Suwon-si (KR); Han Ho SHIN, Anyang-si (KR); Ju-Young PARK, Suwon-si (KR)

(21) Appl. No.: 18/788,074

(22) Filed: Jul. 29, 2024

Related U.S. Application Data
(63) Continuation of application No. 16/646,564, filed on Mar. 11, 2020.

(30) Foreign Application Priority Data
Sep. 12, 2017 (KR) 10-2017-0116497

(51) Int. Cl. A61K 31/415; A61P 1/16; A61P 19/04

(52) U.S. Cl. CPC A61K 31/415; A61P 1/16; A61P 19/04

(57) ABSTRACT

The present inventive concept relates to a pharmaceutical composition for prevention or treatment of fibrosis or a health functional food composition for prevention or alleviation of fibrosis, each composition containing a 1H-pyrazole-3-amide-based compound or a pharmaceutically acceptable salt thereof as an active ingredient. The compound exhibits a fibrosis mitigation effect in fibrosis-induced mouse models, and thus the compositions containing the compound as an active ingredient can be favorably utilized for prevention or treatment of fibrosis.

This application is a continuation of U.S. patent application Ser. No. 16/646,564, filed Mar. 11, 2020, which is a 35 U.S.C. § 371 National Stage patent application of International patent application PCT/KR2018/010671, filed on Sep. 12, 2018, which claims the benefit of Korean Patent Applications No. 10-2017-0116497 filed on Sep. 12, 2017, which are hereby incorporated by references.

### TECHNICAL FIELD

The present disclosure relates to a pharmaceutical composition for preventing or treating fibrosis or a health functional food composition for preventing or alleviating fibrosis, the composition comprising a 1H-pyrazole-3-amide based compound derivative.

### BACKGROUND ART

Fibrosis, as a disease in which abnormal generation, accumulation and deposition of an extracellular matrix are occurred by fibroblast, is referred to as abnormal accumulation of a collagen matrix caused by injury or inflammation which changes structure and function of various tissues. Most disease causes of fibrosis include excessive accumulation of a collagen matrix replacing normal tissues irrespective of an outbreak place of fibrosis. Particularly, fibrosis caused in kidney, liver, lung, heart, fat, bone or bone marrow, fat, and skin induces dysfunction of organs and may lead to death in the worst case. The fibroblast performs a function of forming fibrous tissues by producing a precursor of the extracellular matrix in a normal state. The extracellular matrix, i.e., material between connective tissue cells exists in a protein form such as fibronectin, laminin, chondronectin or collagen.

Meanwhile, development of fibrosis is associated with excessive expression and excessive production of TGF-β (Transforming growth factor-β) in tissues, and fibrosis in the skin may induce formation of hypertrophic scar or keloid causing a serious problem in growth and development, function, and movement of tissues. Actually, expression of TGF-β and its receptor is exceptionally increased in fibroblast, keloid fibroblast or the like in which hypertrophic scar is occurred.

TGF-β (Transforming growth factor-β) is cytokine having various functions of controlling many biological reactions including growth and differentiation, apoptosis, movement, adhesion, angiogenesis, wound healing, and others of cells. TGF-β in fibroblast of the skin stimulates growth and movement of the cells, and further induces expression and deposition of many extracellular matrix components including a type 1 collagen which is most abundant in the skin. Furthermore, when TGF-β signal transduction is increased, fibrosis, i.e., a complex tissue disease originated from excessive accumulation of an extracellular matrix may occur.

Such a TGF-β has been known to be involved in causes of various diseases. A decrease in TGF-β induces atherosclerosis, while an increase in TGF-β is a cause of various types of fibrosis. Although the relationship between cause and effect of TGF-β has been well established in causes of fibrosis, little has been reported on its mechanism in which TGF-β induces fibrosis after passing through any processes. Further, although fibrosis is commonly related to oxidative stress, its mechanism has not also been known.

### DISCLOSURE

### Technical Problem

An objective of the present inventive concept is to provide a pharmaceutical composition which can effectively prevent or treat fibrosis.

The other objective of the present inventive concept is to provide a health functional food composition which can effectively prevent or ameliorate fibrosis.

### Technical Solution

In order to achieve the above objective, the present inventive concept provides a pharmaceutical composition for preventing or treating fibrosis, the composition comprising a 1 H-pyrazole-3-am ide based compound represented by the following chemical formula 1 or a pharmaceutically acceptable salt thereof as an active ingredient.

In order to achieve the other objective, the present inventive concept provides a health functional food composition for preventing or ameliorating fibrosis, the composition comprising a 1H-pyrazole-3-amide based compound represented by the following chemical formula 1 or a pharmaceutically acceptable salt thereof as an active ingredient:

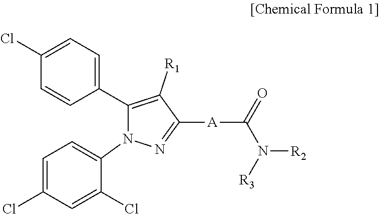

In chemical formula 1, A is

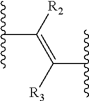

or

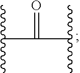 [SMILES: *C(*)=O]

R₁ is hydrogen, a (C₁-C₁₀) alkyl, a (C₃-C₇) cycloalkyl, a (C₆-C₂₀) aryl, or a (C₆-C₂₀)ar(C₁-C₁₀) alkyl; R₂ and R₃ are independently hydrogen, a (C₁-C₁₀) alkyl, a (C₆-C₂₀) aryl, a (C₆-C₂₀)ar(C₁-C₁₀) alkyl, a (C₃-C₇) cycloalkyl, a (C₃-C₇) heterocycloalkyl including one or more heteroatoms selected from N, O and S, adamantyl, piperidino, or pyrrolidino, R₂ and R₃ are connected by a (C₃-C₇) alkylene such that a ring can be formed, and one or more carbon atoms of the alkylene can be substituted by NR₆, O or S; R₄ and R₅ are each independently hydrogen, a (C₁-C₁₀) alkyl, a (C₃-C₇) cycloalkyl, or a (C₆-C₂₀) aryl; R₆ is hydrogen, a (C₁-C₁₀) alkyl, or a (C₁-C₁₀) alkoxycarbonyl; alkyl, aryl, aralkyl, cycloalkyl, adamantyl or heterocycloalkyl of the R₁, R₂ and R₃ may be further substituted by one or more substituents selected from halogen, a (C₁-C₁₀) alkyl, a halo (C₁-C₁₀) alkyl, a (C₁-C₁₀) alkoxy, a mono or di-(C₁-C₁₀) alkylamino, a mono or di-(C₆-C₂₀) arylamino, a (C₃-C₇) cycloalkyl, a (C₂-C₂₀) heteroaryl, hydroxy, a (C₃-C₇) heterocycloalkyl including one or more heteroatoms selected from N, O and S, pyrrolidino, and piperidino; however, R₂ and R₃ are not hydrogen at the same time.

### Advantageous Effects

1H-pyrazole-3-amide based compound derivatives according to the present inventive concept effectively inhibits fibrosis in adipose tissues and hepatic tissues of fibrosis model mice. Therefore, the 1H-pyrazole-3-amide based compound derivatives can be utilized as a pharmaceutical composition or a health functional food composition which can prevent, treat or ameliorate fibrosis in various tissues.

### DESCRIPTION OF DRAWINGS

FIG. 1 shows a structural formula of a 1H-pyrazole-3-amide based compound used in an embodiment of the present inventive concept.

FIG. 2 is results of confirming fibrosis treatment effects of compounds according to the present inventive concept by executing a Picrosirius staining method on adipose tissues of fibrosis model mice.

FIG. 3 is results of confirming fibrosis treatment effects of compounds according to the present inventive concept by executing a quantitative real-time PCR method on adipose tissues of fibrosis model mice.

FIG. 4 is results of confirming fibrosis treatment effects of compounds according to the present inventive concept by executing a Picrosirius staining method on hepatic tissues of fibrosis model mice.

FIG. 5 is results of confirming fibrosis treatment effects of compounds according to the present inventive concept by executing a quantitative real-time PCR method on hepatic tissues of fibrosis model mice.

### DETAILED DESCRIPTION

Hereinafter, the present inventive concept will be described more in detail.

The present inventive concept provides a pharmaceutical composition for preventing or treating fibrosis, the composition comprising a 1H-pyrazole-3-amide based compound represented by the following chemical formula 1 or a pharmaceutically acceptable salt thereof as an active ingredient:

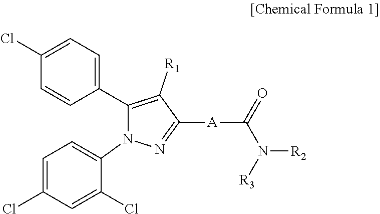

In chemical formula 1, A is

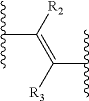

or

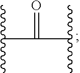 [SMILES: *C(*)=O]

or R₁ is hydrogen, a (C₁-C₁₀) alkyl, a (C₃-C₇) cycloalkyl, a (C₆-C₂₀) aryl, or a (C₆-C₂₀)ar(C₁-C₁₀) alkyl; R₂ and R₃ are independently hydrogen, a (C₁-C₁₀) alkyl, a (C₆-C₂₀) aryl, a (C₆-C₂₀)ar(C₁-C₁₀) alkyl, a (C₃-C₇) cycloalkyl, a (C₃-C₇) heterocycloalkyl including one or more heteroatoms selected from N, O and S, adamantyl, piperidino, or pyrrolidino, R₂ and R₃ are connected by a (C₃-C₇) alkylene such that a ring can be formed, and carbon atoms of the alkylene can be substituted by one or more of NR₆, O and S; R₄ and R₅ are each independently hydrogen, a (C₁-C₁₀) alkyl, a (C₃-C₇) cycloalkyl, or a (C₆-C₂₀) aryl; R₆ is hydrogen, a (C₁-C₁₀) alkyl, or a (C₁-C₁₀) alkoxycarbonyl; alkyl, aryl, aralkyl, cycloalkyl, adamantyl or heterocycloalkyl of the R₁, R₂ and R₃ may be further substituted by one or more substituents selected from halogen, a (C₁-C₁₀) alkyl, a halo (C₁-C₁₀) alkyl, a (C₁-C₁₀) alkoxy, a mono or di-(C₁-C₁₀) alkylamino, a mono or di-(C₆-C₂₀) arylamino, a (C₃-C₇) cycloalkyl, a (C₂-C₂₀) heteroaryl, hydroxy, a (C₃-C₇) heterocycloalkyl including one or more heteroatoms selected from N, O and S, pyrrolidino, and piperidino; however, R₂ and R₃ are not hydrogen at the same time.

Specifically, in chemical formula 1, R₁ is a (C₁-C₁₀) alkyl; R₂ and R₃ are each independently hydrogen, a (C₁-C₁₀) alkyl, a (C₆-C₂₀) aryl, a (C₆-C₂₀)ar(C₁-C₁₀) alkyl, a (C₃-C₇) cycloalkyl, piperidino, adamantyl and piperidinyl, or R₂ and R₃ are connected by a (C₄-C₆) alkylene such that a ring can be formed, and carbon atoms of the alkylene can be substituted by one or more of NR₆, O and S; R₄ and R₅ are hydrogen; R₆ is hydrogen, a (C₁-C₁₀) alkyl, or a (C₁-C₁₀) alkoxycarbonyl; alkyl, aryl, or aralkyl of the R₂ and R₃ may be further substituted by one or more substituents selected from halogen, a (C₁-C₁₀) alkyl, a halo (C₁-C₁₀) alkyl, a (C₁-C₁₀) alkoxy, a mono or di-(C₁-C₁₀) alkylamino, a (C₃-C₇) cycloalkyl, a (C₂-C₂₀) heteroaryl, and hydroxy.

Preferably, the 1H-pyrazole-3-amide based compound may be selected from the group consisting of: (E)-N-(4-chlorophenyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-phenyl acrylamide; (E)-N-(3-chlorobenzyl)-3-(5-(4-chlorophenyl)-1-(2,4-dichlorophenyl)-4-methyl-1H-pyrazole-3-yl)acrylamide; (E)-N-(2-chlorobenzyl)-3-(5-(4-chlorophenyl)-1-(2,4-dichlorophenyl)-4-methyl-1H-pyrazole-3-yl)acrylamide; (E)-N-benzyl-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(4-methoxybenzyl)acrylamide; (E)-N-(4-chlorobenzyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(4-fluorobenzyl)acrylamide; (E)-3-(5-(4-chlorophenyl)-1-(2,4-dichlorophenyl)-4-methyl-1H-pyrazole-3-yl)-N-(3-fluorobenzyl)acrylamide; (E)-3-(5-(4-chlorophenyl)-1-(2,4-dichlorophenyl)-4-methyl-1H-pyrazole-3-yl)-N-isopropylacrylamide; (E)-3-(5-(4-chlorophenyl)-1-(2,4-dichlorophenyl)-4-methyl-1H-pyrazole-3-yl)-N-(3-chloropropyl)acrylamide; (E)-3-(5-(4-chlorophenyl)-1-(2,4-dichlorophenyl)-4-methyl-1H-pyrazole-3-yl)-N-hexylacrylamide; (E)-3-(5-(4-chlorophenyl)-1-(2,4-dichlorophenyl)-4-methyl-1H-pyrazole-3-yl)-N-(cyclohexylmethyl)acrylamide; (E)-3-(5-(4-chlorophenyl)-1-(2,4-dichlorophenyl)-4-methyl-1H-pyrazole-3-yl)-N-(3-(trifluoromethyl)benzyl)acrylamide; (E)-3-(5-(4-chlorophenyl)-1-(2,4-dichlorophenyl)-4-methyl-1H-pyrazole-3-yl)-N-(4-(trifluoromethyl)benzyl)acrylamide; (E)-3-(5-(4-chlorophenyl)-1-(2,4-dichlorophenyl)-4-methyl-1H-pyrazole-3-yl)-N-(4-(dimethylamino)benzyl)acrylamide; (E)-N-(3-chlorophenethyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(piperidine-1-yl)acrylamide; (E)-3-(5-(4-chlorophenyl)-1-(2,4-dichlorophenyl)-4-methyl-1H-pyrazole-3-yl)-N-(furan-3-yl methyl)acrylamide; (E)-4-(5-(4-chlorophenyl)-1-(2,4-dichlorophenyl)-4-methyl-1H-pyrazole-3-yl)-1-(piperidine-4-yl)acrylamide; 2-(5-(4-chlorophenyl)-1-(2,4-dichlorophenyl)-4-methyl-1H-pyrazole-3-yl)-2-oxo-N-phenyl acetamide; 2-(5-(4-chlorophenyl)-1-(2,4-dichlorophenyl)-4-methyl-1H-pyrazole-3-yl)-N-methyl-2-oxo-N-phenyl acetamide; N-(3-chlorobenzyl)-2-(5-(4-chlorophenyl)-1-(2,4-dichlorophenyl)-4-methyl-1H-pyrazole-3-yl)-2-oxo-acetamide; N-(3-chlorophenethyl)-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-N-cyclopentyl-2-oxo-acetamide; 2-(5-(4-chlorophenyl)-1-(2,4-dichlorophenyl)-4-methyl-1H-pyrazole-3-yl)-N-cyclohexyl-2-oxo-acetamide; 2-(5-(4-chlorophenyl)-1-(2,4-dichlorophenyl)-4-methyl-1H-pyrazole-3-yl)-N-cycloheptyl-2-oxo-acetamide; 1-(5-(4-chlorophenyl)-1-(2,4-dichlorophenyl)-4-methyl-1H-pyrazole-3-yl)-2-(pyrrolidine-1-yl)ethane-1,2-dione; 1-(5-(4-chlorophenyl)-1-(2,4-dichlorophenyl)-4-methyl-1H-pyrazole-3-yl)-2-(piperidine-1-yl)ethane-1,2-dione; 1-(azepane-1-yl)-2-(5-(4-chlorophenyl)-1-(2,4-dichlorophenyl)-4-methyl-1H-pyrazole-3-yl)ethane-1,2-dione; 2-(5-(4-chlorophenyl)-1-(2,4-dichlorophenyl)-4-methyl-1H-pyrazole-3-yl)-2-oxo-N-(piperidine-1-yl)acetamide; 2-(5-(4-chlorophenyl)-1-(2,4-dichlorophenyl)-4-methyl-1H-pyrazole-3-yl)-N-(cyclohexylmethyl)-2-oxo-acetamide; 1-(5-(4-chlorophenyl)-1-(2,4-dichlorophenyl)-4-methyl-1H-pyrazole-3-yl)-2-(piperazine-1-yl)ethane-1,2-dione; tert-butyl 4-(2-(5-(4-chlorophenyl)-1-(2,4-dichlorophenyl)-4-methyl-1H-pyrazole-3-yl)-2-oxoacetyl)piperazine-1-carboxylate; 1-(5-(4-chlorophenyl)-1-(2,4-dichlorophenyl)-4-methyl-1H-pyrazole-3-yl)-2-(4-methylpiperazine-1-yl)ethane-1,2-dione; 1-(5-(4-chlorophenyl)-1-(2,4-dichlorophenyl)-4-methyl-1H-pyrazole-3-yl)-2-morpholinoethane-1,2-dione; 1-(5-(4-chlorophenyl)-1-(2,4-dichlorophenyl)-4-methyl-1H-pyrazole-3-yl)-2-thiomorpholinoethane-1,2-dione; 2-(5-(4-chlorophenyl)-1-(2,4-dichlorophenyl)-4-methyl-1H-pyrazole-3-yl)-N-adamantyl-2-oxo-acetamide; 2-(5-(4-chlorophenyl)-1-(2,4-dichlorophenyl)-4-methyl-1H-pyrazole-3-yl)-N,N-dicyclohexyl-2-oxo-acetamide; 2-(5-(4-chlorophenyl)-1-(2,4-dichlorophenyl)-4-methyl-1H-pyrazole-3-yl)-N,N-dihexyl-2-oxo-acetamide; 2-(5-(4-chlorophenyl)-1-(2,4-dichlorophenyl)-4-methyl-1H-pyrazole-3-yl)-N-ethyl-2-oxo-acetamide; 2-(5-(4-chlorophenyl)-1-(2,4-dichlorophenyl)-4-methyl-1H-pyrazole-3-yl)-2-oxo-N-propyl acetamide; 2-(5-(4-chlorophenyl)-1-(2,4-dichlorophenyl)-4-methyl-1H-pyrazole-3-yl)-N-isopropyl-2-oxo-acetamide; N-butyl-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-N-isobutyl-2-oxo-acetamide; N-tert-butyl-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-2-oxo-N-pentylacetacetamide; 2-(5-(4-chlorophenyl)-1-(2,4-dichlorophenyl)-4-methyl-1H-pyrazole-3-yl)-N-hexyl-2-oxo-acetamide; 2-(5-(4-chlorophenyl)-1-(2,4-dichlorophenyl)-4-methyl-1H-pyrazole-3-yl)-N-heptyl-2-oxo-acetamide; 2-(5-(4-chlorophenyl)-1-(2,4-dichlorophenyl)-4-methyl-1H-pyrazole-3-yl)-N-octyl-2-oxo-acetamide; and 2-(5-(4-chlorophenyl)-1-(2,4-dichlorophenyl)-4-methyl-1H-pyrazole-3-yl)-N-(2-hydroxyethyl)-2-oxo-acetamide.

More preferably, the 1H-pyrazole-3-am ide based compound may be (E)-N-(3-chlorobenzyl)-3-(5-(4-chlorophenyl)-1-(2,4-dichlorophenyl)-4-methyl-1H-pyrazole-3-yl)acrylamide or 2-(5-(4-chlorophenyl)-1-(2,4-dichlorophenyl)-4-methyl-1H-pyrazole-3-yl)-N-(2-hydroxyethyl)-2-oxo-acetamide. Therefore, as the above-mentioned two compounds exhibit excellent fibrosis alleviating effects in fibrosis model mice, the compounds can be utilized as a pharmaceutical composition for preventing or treating fibrosis.

At this time, although fibrosis may be induced in one or more tissues selected from the group consisting of kidney, liver, lung, heart, bone, bone marrow, fat, and skin, the present inventive concept is not limited thereto if the tissues are tissues of enabling fibrosis to occur.

In an embodiment of the present inventive concept, a pharmaceutical composition for preventing or treating fibrosis, the pharmaceutical composition comprising the 1H-pyrazole-3-amide based compound as an active ingredient may be formed in any one formylation selected from the group consisting of injection, granule, powder, tablet, pill, capsule, suppository, gel, suspension, emulsion, drop, or liquid according to a conventional method.

In another specific embodiment, one or more additives selected from the group consisting of appropriate carrier, excipient, disintegrating agent, sweetener, coating agent, expansion agent, lubricant, slip modifier, flavoring agent, antioxidant, buffer solution, bacteriostat, diluent, dispersant, surfactant, and binder which are commonly used may be additionally included in the preparation of a pharmaceutical composition for preventing or treating fibrosis, the pharmaceutical composition comprising the 1H-pyrazole-3-amide based compound as an active ingredient.

Specifically, the carrier, excipient and diluent may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil, a solid formulation for oral administration includes a tablet, a pill, a powder, a granule, capsule and others, and such a solid formulation may be prepared by mixing the composition with at least one excipient, e.g., starch, calcium carbonate, sucrose or lactose, gelatin and others. Further, the solid formulation for oral administration may also include lubricants such as magnesium stearate and talc in addition to a simple excipient. A liquid formulation for oral administration includes a suspension, a liquid medicine, an emulsion, a syrup and others, and may include various excipients such as a wetting agent, a sweetener, an aromatic agent, a preserving agent, and others in addition to water, liquid paraffin that are a frequently-used simple diluent. A formulation for parenteral administration includes a sterilized aqueous solution, a non-aqueous solvent, a suspension, an emulsion, a freeze-dried preparation, a suppository, and others. The non-aqueous solvent and the suspension may include vegetable oil such as propylene glycol, polyethylene glycol or olive oil, an injectable ester such as ethyl oleate, and others. A base material of the suppository may include witepsol, macrogol, tween 61, cacao butter, laurin butter, glycerogelatin, and others.

The pharmaceutical composition according to an embodiment of the present inventive concept can be administered to an object in the usual way through intravenous, intra-arterial, intraperitoneal, intramuscular, intrasternal, percutaneous, nasal, inhalational, topical, rectal, oral, intraocular, or intradermal pathway.

A preferred administration dose of the 1H-pyrazole-3-amide based compound may be varied depending on conditions and weight of the object, type and severity of disease, formulation type, administration route and administration period, and may be determined adequately by those skilled in the art. Administration may be made once or several times a day, and the scope of the present inventive concept is not limited thereby.

In the present inventive concept, although the above-mentioned ‘object’ may be mammals including human, the present inventive concept is not limited thereto.

Furthermore, the present inventive concept provides a health functional food composition for preventing or ameliorating fibrosis, the composition comprising a 1H-pyrazole-3-amide based compound represented by the following chemical formula 1or a pharmaceutically acceptable salt thereof as an active ingredient:

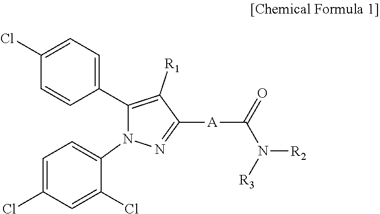

In chemical formula 1, A is

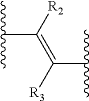

or

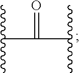 [SMILES: *C(*)=O]

or R₁ is hydrogen, a (C₁-C₁₀) alkyl, a (C₃-C₇) cycloalkyl, a (C₆-C₂₀) aryl, or a (C₆-C₂₀) ar (C₁-C₁₀) alkyl; R₂ and R₃ are independently hydrogen, a (C₁-C₁₀) alkyl, a (C₆-C₂₀) aryl, a (C₆-C₂₀) ar (C₁-C₁₀) alkyl, a (C₃-C₇) cycloalkyl, a (C₃-C₇) heterocycloalkyl including one or more heteroatoms selected from N, O and S, adamantyl, piperidino, or pyrrolidino, R₂ and R₃ are connected by a (C₃-C₇) alkylene such that a ring can be formed, and carbon atoms of the alkylene can be substituted by one or more of NR₆, O and S; R₄ and R₅ are each independently hydrogen, a (C₁-C₁₀) alkyl, a (C₃-C₇) cycloalkyl, or a (C₆-C₂₀) aryl; R₆ is hydrogen, a (C₁-C₁₀) alkyl, or a (C₁-C₁₀) alkoxycarbonyl; alkyl, aryl, aralkyl, cycloalkyl, adamantyl or heterocycloalkyl of the R₁, R₂ and R₃ may be further substituted by one or more substituents selected from halogen, a (C₁-C₁₀) alkyl, a halo (C₁-C₁₀) alkyl, a (C₁-C₁₀) alkoxy, a mono or di-(C₁-C₁₀) alkylamino, a mono or di-(C₆-C₂₀) arylamino, a (C₃-C₇) cycloalkyl, a (C₂-C₂₀) heteroaryl, hydroxy, a (C₃-C₇) heterocycloalkyl including one or more heteroatoms selected from N, O and S, pyrrolidino, and piperidino; however, R₂ and R₃ are not hydrogen at the same time.

Specifically, in chemical formula 1, R₁ is a (C₁-C₁₀) alkyl; R₂ and R₃ are each independently hydrogen, a (C₁-C₁₀) alkyl, a (C₆-C₂₀) aryl, a (C₆-C₂₀) ar (C₁-C₁₀) alkyl, a (C₃-C₇) cycloalkyl, piperidino, adamantyl and piperidinyl, or R₂ and R₃ are connected by a (C₄-C₆) alkylene such that a ring can be formed, and carbon atoms of the alkylene can be substituted by one or more of NR₆, O and S; R₄ and R₅ are hydrogen; R₆ is hydrogen, a (C₁-C₁₀) alkyl, or a (C₁-C₁₀) alkoxycarbonyl; alkyl, aryl, or aralkyl of the R₂ and R₃ may be further substituted by one or more substituents selected from halogen, a (C₁-C₁₀) alkyl, a halo (C₁-C₁₀) alkyl, a (C₁-C₁₀) alkoxy, a mono or di-(C₁-C₁₀) alkylamino, a (C₃-C₇) cycloalkyl, a (C₂-C₂₀) heteroaryl, and hydroxy.

Preferably, the 1H-pyrazole-3-amide based compound may be selected from the group consisting of: (E)-N-(4-chlorophenyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-phenyl acrylamide; (E)-N-(3-chlorobenzyl)-3-(5-(4-chlorophenyl)-1-(2,4-dichlorophenyl)-4-methyl-1H-pyrazole-3-yl)acrylamide; (E)-N-(2-chlorobenzyl)-3-(5-(4-chlorophenyl)-1-(2,4-dichlorophenyl)-4-methyl-1H-pyrazole-3-yl)acrylamide; (E)-N-benzyl-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(4-methoxybenzyl)acrylamide; (E)-N-(4-chlorobenzyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(4-fluorobenzyl)acrylamide; (E)-3-(5-(4-chlorophenyl)-1-(2,4-dichlorophenyl)-4-methyl-1H-pyrazole-3-yl)-N-(3-fluorobenzyl)acrylamide; (E)-3-(5-(4-chlorophenyl)-1-(2,4-dichlorophenyl)-4-methyl-1H-pyrazole-3-yl)-N-isopropylacrylamide; (E)-3-(5-(4-chlorophenyl)-1-(2,4-dichlorophenyl)-4-methyl-1H-pyrazole-3-yl)-N-(3-chloropropyl)acrylamide; (E)-3-(5-(4-chlorophenyl)-1-(2,4-dichlorophenyl)-4-methyl-1H-pyrazole-3-yl)-N-hexylacrylamide; (E)-3-(5-(4-chlorophenyl)-1-(2,4-dichlorophenyl)-4-methyl-1H-pyrazole-3-yl)-N-(cyclohexylmethyl)acrylamide; (E)-3-(5-(4-chlorophenyl)-1-(2,4-dichlorophenyl)-4-methyl-1H-pyrazole-3-yl)-N-(3-(trifluoromethyl)benzyl)acrylamide; (E)-3-(5-(4-chlorophenyl)-1-(2,4-dichlorophenyl)-4-methyl-1H-pyrazole-3-yl)-N-(4-(trifluoromethyl)benzyl)acrylamide; (E)-3-(5-(4-chlorophenyl)-1-(2,4-dichlorophenyl)-4-methyl-1H-pyrazole-3-yl)-N-(4-(dimethylamino)benzyl)acrylamide; (E)-N-(3-chlorophenethyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(piperidine-1-yl)acrylamide; (E)-3-(5-(4-chlorophenyl)-1-(2,4-dichlorophenyl)-4-methyl-1H-pyrazole-3-yl)-N-(furan-3-yl methyl)acrylamide; (E)-4-(5-(4-chlorophenyl)-1-(2,4-dichlorophenyl)-4-methyl-1H-pyrazole-3-yl)-1-(piperidine-4-yl)acrylamide; 2-(5-(4-chlorophenyl)-1-(2,4-dichlorophenyl)-4-methyl-1H-pyrazole-3-yl)-2-oxo-N-phenyl acetamide; 2-(5-(4-chlorophenyl)-1-(2,4-dichlorophenyl)-4-methyl-1H-pyrazole-3-yl)-N-methyl-2-oxo-N-phenyl acetamide; N-(3-chlorobenzyl)-2-(5-(4-chlorophenyl)-1-(2,4-dichlorophenyl)-4-methyl-1H-pyrazole-3-yl)-2-oxo-acetamide; N-(3-chlorophenethyl)-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-N-cyclopentyl-2-oxo-acetamide; 2-(5-(4-chlorophenyl)-1-(2,4-dichlorophenyl)-4-methyl-1H-pyrazole-3-yl)-N-cyclohexyl-2-oxo-acetamide; 2-(5-(4-chlorophenyl)-1-(2,4-dichlorophenyl)-4-methyl-1H-pyrazole-3-yl)-N-cycloheptyl-2-oxo-acetamide; 1-(5-(4-chlorophenyl)-1-(2,4-dichlorophenyl)-4-methyl-1H-pyrazole-3-yl)-2-(pyrrolidine-1-yl)ethane-1,2-dione; 1-(5-(4-chlorophenyl)-1-(2,4-dichlorophenyl)-4-methyl-1H-pyrazole-3-yl)-2-(piperidine-1-yl)ethane-1,2-dione; 1-(azepane-1-yl)-2-(5-(4-chlorophenyl)-1-(2,4-dichlorophenyl)-4-methyl-1H-pyrazole-3-yl)ethane-1,2-dione; 2-(5-(4-chlorophenyl)-1-(2,4-dichlorophenyl)-4-methyl-1H-pyrazole-3-yl)-2-oxo-N-(piperidine-1-yl)acetamide; 2-(5-(4-chlorophenyl)-1-(2,4-dichlorophenyl)-4-methyl-1H-pyrazole-3-yl)-N-(cyclohexylmethyl)-2-oxo-acetamide; 1-(5-(4-chlorophenyl)-1-(2,4-dichlorophenyl)-4-methyl-1H-pyrazole-3-yl)-2-(piperazine-1-yl)ethane-1,2-dione; tert-butyl 4-(2-(5-(4-chlorophenyl)-1-(2,4-dichlorophenyl)-4-methyl-1H-pyrazole-3-yl)-2-oxoacetyl)piperazine-1-carboxylate; 1-(5-(4-chlorophenyl)-1-(2,4-dichlorophenyl)-4-methyl-1H-pyrazole-3-yl)-2-(4-methylpiperazine-1-yl)ethane-1,2-dione; 1-(5-(4-chlorophenyl)-1-(2,4-dichlorophenyl)-4-methyl-1H-pyrazole-3-yl)-2-morpholinoethane-1,2-dione; 1-(5-(4-chlorophenyl)-1-(2,4-dichlorophenyl)-4-methyl-1H-pyrazole-3-yl)-2-thiomorpholinoethane-1,2-dione; 2-(5-(4-chlorophenyl)-1-(2,4-dichlorophenyl)-4-methyl-1H-pyrazole-3-yl)-N-adamantyl-2-oxo-acetamide; 2-(5-(4-chlorophenyl)-1-(2,4-dichlorophenyl)-4-methyl-1H-pyrazole-3-yl)-N,N-dicyclohexyl-2-oxo-acetamide; 2-(5-(4-chlorophenyl)-1-(2,4-dichlorophenyl)-4-methyl-1H-pyrazole-3-yl)-N,N-dihexyl-2-oxo-acetamide; 2-(5-(4-chlorophenyl)-1-(2,4-dichlorophenyl)-4-methyl-1H-pyrazole-3-yl)-N-ethyl-2-oxo-acetamide; 2-(5-(4-chlorophenyl)-1-(2,4-dichlorophenyl)-4-methyl-1H-pyrazole-3-yl)-2-oxo-N-propyl acetamide; 2-(5-(4-chlorophenyl)-1-(2,4-dichlorophenyl)-4-methyl-1H-pyrazole-3-yl)-N-isopropyl-2-oxo-acetamide; N-butyl-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-N-isobutyl-2-oxo-acetamide; N-tert-butyl-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-2-oxo-N-pentylacetacetamide; 2-(5-(4-chlorophenyl)-1-(2,4-dichlorophenyl)-4-methyl-1H-pyrazole-3-yl)-N-hexyl-2-oxo-acetamide; 2-(5-(4-chlorophenyl)-1-(2,4-dichlorophenyl)-4-methyl-1H-pyrazole-3-yl)-N-heptyl-2-oxo-acetamide; 2-(5-(4-chlorophenyl)-1-(2,4-dichlorophenyl)-4-methyl-1H-pyrazole-3-yl)-N-octyl-2-oxo-acetamide; and 2-(5-(4-chlorophenyl)-1-(2,4-dichlorophenyl)-4-methyl-1H-pyrazole-3-yl)-N-(2-hydroxyethyl)-2-oxo-acetamide.

More preferably, the 1H-pyrazole-3-amide based compound may be (E)-N-(3-chlorobenzyl)-3-(5-(4-chlorophenyl)-1-(2,4-dichlorophenyl)-4-methyl-1H-pyrazole-3-yl)acrylamide or 2-(5-(4-chlorophenyl)-1-(2,4-dichlorophenyl)-4-methyl-1H-pyrazole-3-yl)-N-(2-hydroxyethyl)-2-oxo-acetamide. Therefore, as the above-mentioned two compounds exhibit excellent fibrosis alleviating effects in fibrosis model mice, the compounds can be utilized as a health functional food composition for preventing or ameliorating fibrosis.

At this time, although fibrosis may be induced in one or more tissues selected from the group consisting of kidney, liver, lung, heart, bone, bone marrow, fat, and skin, the present inventive concept is not limited thereto if the tissues are tissues of enabling fibrosis to generate.

In an embodiment of the present inventive concept, the health functional food composition may comprise various nutritional supplements, vitamins, minerals (electrolytes), flavoring agents such as synthetic flavoring agents and natural flavoring agents, coloring agents, enhancers (cheese, chocolate, etc.), pectic acid and its salt, alginic acid and its salt, organic acids, protective colloidal thickeners, pH adjusters, stabilizers, preservatives, glycerin, alcohols, and carbonating agents used in carbonated beverages, etc. Moreover, the health functional food composition may comprise fruit flesh for the preparation of natural fruit juices, synthetic fruit juices, and vegetable beverages. Such ingredients may be used alone or in combination. Further, the health functional food composition may be formed in any one form of meats, sausages, breads, chocolates, candies, snacks, cookies, pizza, ramen, gums, ice creams, soups, beverages, teas, functional water, health drinks, alcoholic beverages, and vitamin complexes.

Further, the health functional food composition may further comprise food additives, whether or not the food additives are appropriate as “food additives” is determined by specifications and criteria for corresponding items according to general rules, general test methods and the like of Korean Food Additives Codex approved by Korea Food & Drug Administration unless otherwise stipulated.

For example, items listed in the above-mention “Korean Food Additives Codex” may include chemical synthetics such as ketones, glycine, potassium citrate, nicotinic acid, cinnamic acid and the like, natural additives such as persimmon color, Glycyrrhiza extract, crystal cellulose, Kaoliang color, guar gum and the like, mixed formulations such as an L-sodium glutamate formulation, alkali agents for noodles, a preservative formulation, a tar color formulation and the like, etc.

### Mode(s) for Carrying Out the Invention

Hereinafter, the Examples of the present inventive concept will be described in detail so that the present inventive concept can be easily realized by those skilled in the art. The present inventive concept may, however, be embodied in different forms and should not be constructed as limited to the Examples set forth herein. Rather, these Examples are provided so that this disclosure will be thorough and complete, and will fully convey the scope of the present inventive concept to those skilled in the art.

### <Synthesis Example 1> Synthesis of 1H-Pyrazole-3-Amide Based Compound Derivative

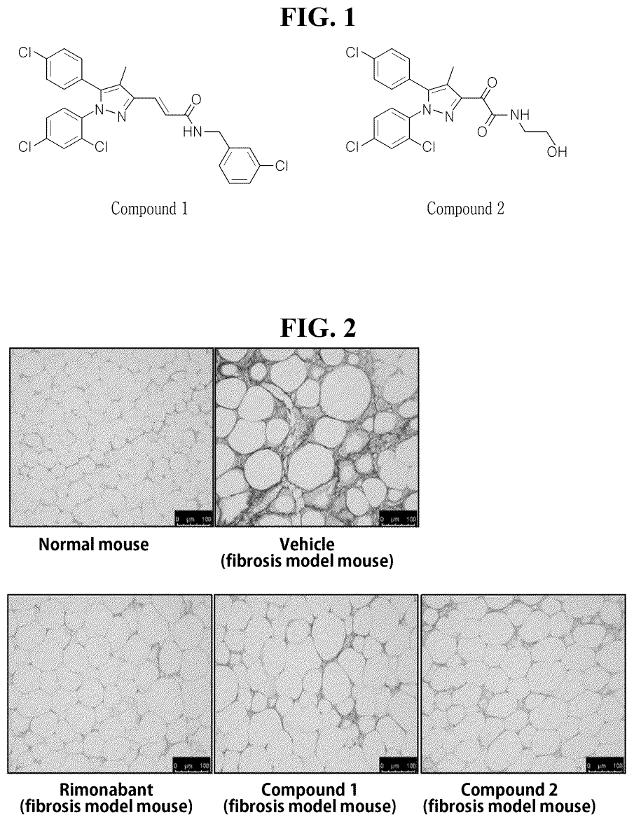

An 1H-pyrazole-3-amide based compound derivative used in the present inventive concept was synthesized and prepared by referring to Korean patent registration No. 10-1070176, thereby performing a method disclosed in the patent. An experiment for confirming fibrosis treatment effects was performed by using (E)-N-(3-chlorobenzyl)-3-(5-(4-chlorophenyl)-1-(2,4-dichlorophenyl)-4-methyl-1H-pyrazole-3-yl)acrylamide (referred to as compound 1) and 2-(5-(4-chlorophenyl)-1-(2,4-dichlorophenyl)-4-methyl-1H-pyrazole-3-yl)-N-(2-hydroxyethyl)-2-oxo-acetamide (referred to as compound 2) among the above-mentioned compounds. At this time, structural formulas of the compounds 1 and 2 are as shown in FIG. 1.

### <Example 1> Conforming Fibrosis Treatment Effects

### (1) Fibrosis Animal Model Construction and Drug Administration

First, after stabilizing 5-week old male C₅₇BL/6 mice for one week, a fibrosis model was constructed by feeding the mice with a high fat diet and a high-carbohydrate diet for 12 weeks, thereby inducing fibrosis of the mice. After that, the mice were divided into total four groups, and a vehicle (negative control group), 10mg/kg of rimonabant (positive control group), 10 mg/kg of a compound 1, and 10mg/kg of a compound 2 were administered to each of the groups by intraperitoneal injection for 4 weeks. In order to perform an experimental process, the mice were sacrificed, and epididymal adipose and liver tissues were extracted from the sacrificed mice.

### (2) Picrosirius Staining

After fixing and embedding the extracted epididymal adipose and liver tissues, paraffin sections were manufactured by slicing the embedded epididymal adipose and liver tissues. After that, fibrosis degrees were analyzed by staining collagen permeated into each of the tissues by using a picrosirius red stain kit.

### (3) Quantitative Real-Time PCR

After separating RNA from the extracted epididymal adipose and liver tissues by using a Trizol reagent (Gibco), cDNA templates were produced by using reverse transcriptase. After injecting such produced cDNA templates into SYBR green dyes by using gene-specific nucleotide primers, PCR products were analyzed to obtain analyzed mRNAs by amplifying the cDNA templates injected into SYBR green dyes with a real-time multiplexing amplification system (CFX connect™, Bio-Rad). At this time, the analyzed mRNAs were αSMA, TGF-β1, COL6A3, and LOX mRNAs, and used primer sequences are as described in the following [Table 1].

TABLE 1

| | | | Sequence |
|---|---|---|---|
| Gene | Type | Base sequence | number |
| αSMA | Forward | TCA GCG CCT CCA GTT CCT | 1 |
| | Reverse | AAA AAA AAC CAC GAG TAA CAA | 2 |
| | | ATCAA | |
| TGF-β1 | Forward | ACAAGG CAC GGG ACC TAT G | 3 |
| | Reverse | TCC CAG TCA GTC CTG GAA ATG | 4 |
| COL6A3 | Forward | TGA CTC ACC TTG TGG TCC TAA | 5 |
| | Reverse | CTT CCC AGA ATC CAG TOT TTC C | 6 |
| LOX | Forward | GTG ATG CTC AGG TAT CCA TCC A | 7 |
| | Reverse | CAC AGT TCT CAAAGC ACA GCG | 8 |

### (4) Experimental Results

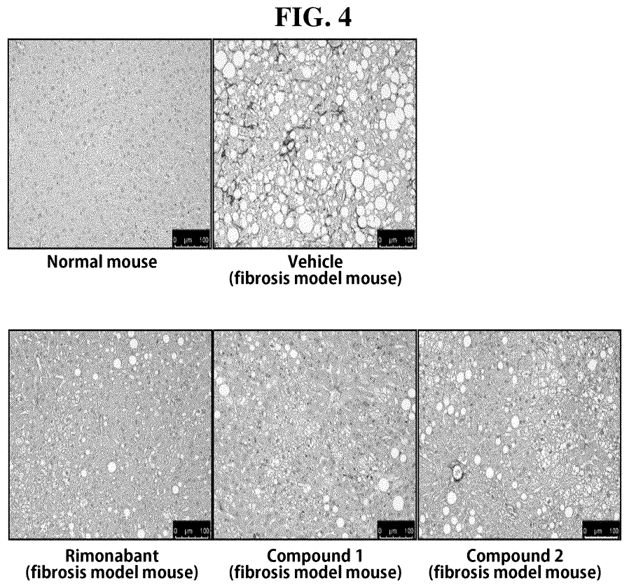

As described above, effects of the compounds 1 and 2 for fibrosis of the epididymal adipose and liver tissues of the mice were confirmed. First, as results of performing picrosirius staining experiments on respective tissues, it can be confirmed that accumulation degrees stained with red color in the respective tissues are reduced compared to a fibrosis-induced negative control group (vehicle treatment group), when treating the compounds 1 and 2 as disclosed in FIG. 2 (adipose tissue) and FIG. 4 (liver tissue).

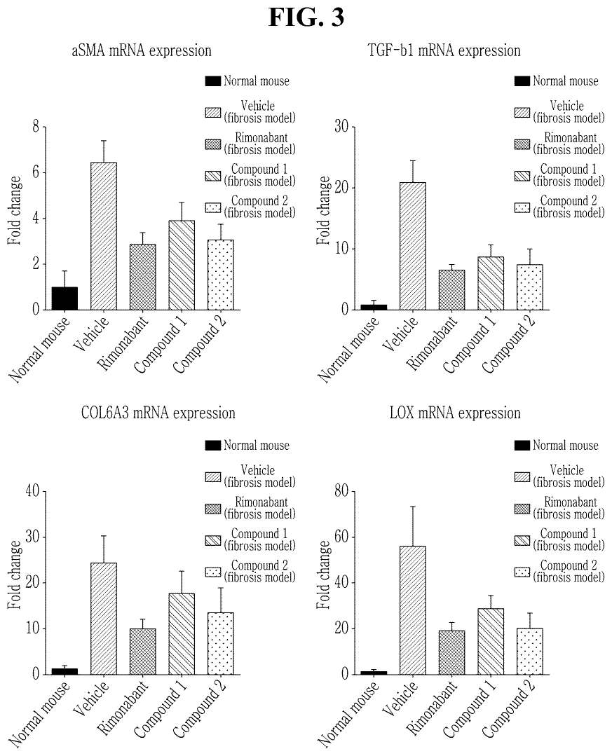

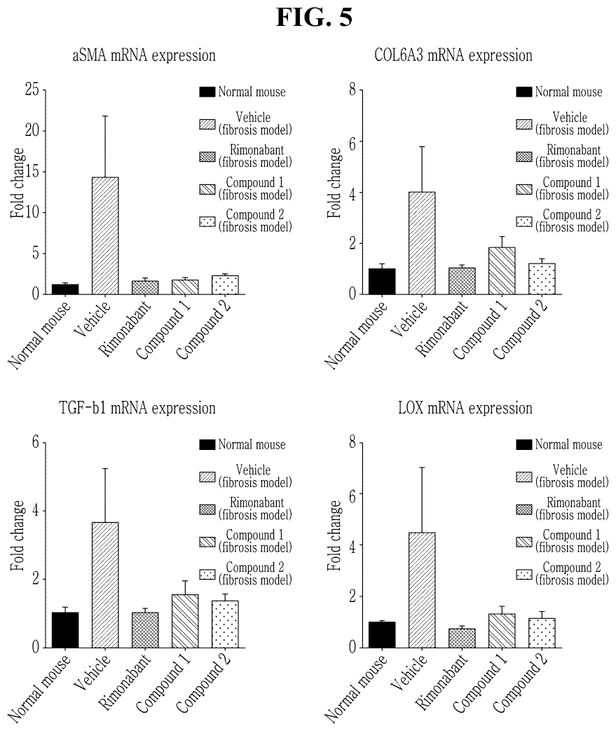

Further, as results of performing quantitative real-time PCR experiments on respective tissues, it can be confirmed that expression of four gene mRNAs is inhibited in an almost similar fashion to when treating rimonabant, i.e., a positive control group in the respective tissues, particularly it is confirmed that an effect of the compound 2 is somewhat high, when treating the compounds 1 and 2 as disclosed in FIG. 3 (adipose tissue) and FIG. 5 (liver tissue).

As described above, as fibrosis symptoms are remarkably reduced when treating the compounds 1 and 2 of the present inventive concept on fibrosis model mice, a composition comprising the compounds as an active ingredient can be usefully utilized as a composition for preventing, ameliorating or treating fibrosis.

Although the present inventive concept has been described along with the accompanying drawings, this is only one of various examples including the gist of the present inventive concept and has an object of enabling a person having ordinary skill in the art to easily practice the invention. Accordingly, it is evident that the present inventive concept is not limited to the aforementioned examples. Accordingly, the range of protection of the present inventive concept should be interpreted based on the following claims, and all of technological spirits within the equivalents of the present inventive concept may fall within the range of right of the present inventive concept by changes, substitutions and replacements without departing from the gist of the present inventive concept. Furthermore, it is evident that the configurations of some drawings have been provided to more clearly describe configurations and have been more exaggerated or reduced than actual configurations.

1. A method of treating fibrosis using a pharmaceutical composition, the composition comprising a 1H-pyrazole-3-amide based compound represented by the following chemical formula 1 or a pharmaceutically acceptable salt thereof as an active ingredient: 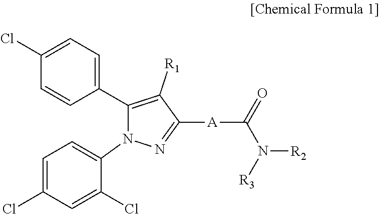
In chemical formula 1, A is 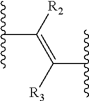
or 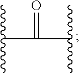 [SMILES: *C(*)=O]
or R₁ is hydrogen, a (C₁-C₁₀) alkyl, a (C₃-C₇) cycloalkyl, a (C₆-C₂₀) aryl, or a (C₆-C₂₀) ar (C₁-C₁₀) alkyl; R₂ and R₃ are independently hydrogen, a (C₁-C₁₀) alkyl, a (C₆-C₂₀) aryl, a (C₆-C₂₀) ar (C₁-C₁₀) alkyl, a (C₃-C₇) cycloalkyl, a (C₃-C₇) heterocycloalkyl including one or more heteroatoms selected from N, O and S, adamantyl, piperidino, or pyrrolidino, R₂ and R₃ are connected by a (C₃-C₇) alkylene such that a ring can be formed, and carbon atoms of the alkylene can be substituted by one or more of NR₆, O and S; R₄ and R₅ are each independently hydrogen, a (C₁-C₁₀) alkyl, a (C₃-C₇) cycloalkyl, or a (C₆-C₂₀) aryl; R₆ is hydrogen, a (C₁-C₁₀) alkyl, or a (C₁-C₁₀) alkoxycarbonyl; alkyl, aryl, aralkyl, cycloalkyl, adamantyl or heterocycloalkyl of the R₁, R₂ and R₃ are further substituted by one or more substituents selected from halogen, a (C₁-C₁₀) alkyl, a halo (C₁-C₁₀) alkyl, a (C₁-C₁₀) alkoxy, a mono or di-(C₁-C₁₀) alkylamino, a mono or di-(C₆-C₂₀) arylamino, a (C₃-C₇) cycloalkyl, a (C₂-C₂₀) heteroaryl, hydroxy, a (C₃-C₇) heterocycloalkyl including one or more heteroatoms selected from N, O and S, pyrrolidino, and piperidino; however, R₂ and R₃ are not hydrogen at the same time.

2. The method of claim 1, wherein, in chemical formula 1, R₁ is a (C₁-C₁₀) alkyl; R₂ and R₃ are each independently hydrogen, a (C₁-C₁₀) alkyl, a (C₆-C₂₀) aryl, a (C₆-C₂₀) ar (C₁-C₁₀) alkyl, a (C₃-C₇) cycloalkyl, piperidino, adamantyl and piperidinyl, or R₂ and R₃ are connected by a (C₄-C₆) alkylene such that a ring can be formed, and carbon atoms of the alkylene can be substituted by one or more of NR₆, O and S; R₄ and R₅ are hydrogen; R₆ is hydrogen, a (C₁-C₁₀) alkyl, or a (C₁-C₁₀) alkoxycarbonyl; alkyl, aryl, or aralkyl of the R₂ and R₃ is further substituted by one or more substituents selected from halogen, a (C₁-C₁₀) alkyl, a halo (C₁-C₁₀) alkyl, a (C₁-C₁₀) alkoxy, a mono or di-(C₁-C₁₀) alkylamino, a (C₃-C₇) cycloalkyl, a (C₂-C₂₀) heteroaryl, and hydroxy.

3. The method of claim 1, wherein the 1H-pyrazole-3-amide based compound is selected from the group consisting of: (E)-N-(4-chlorophenyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-phenyl acrylamide; (E)-N-(3-chlorobenzyl)-3-(5-(4-chlorophenyl)-1-(2,4-dichlorophenyl)-4-methyl-1H-pyrazole-3-yl)acrylamide; (E)-N-(2-chlorobenzyl)-3-(5-(4-chlorophenyl)-1-(2,4-dichlorophenyl)-4-methyl-1H-pyrazole-3-yl)acrylamide; (E)-N-benzyl-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(4-methoxybenzyl)acrylamide; (E)-N-(4-chlorobenzyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(4-fluorobenzyl)acrylamide; (E)-3-(5-(4-chlorophenyl)-1-(2,4-dichlorophenyl)-4-methyl-1H-pyrazole-3-yl)-N-(3-fluorobenzyl)acrylamide; (E)-3-(5-(4-chlorophenyl)-1-(2,4-dichlorophenyl)-4-methyl-1H-pyrazole-3-yl)-N-isopropylacrylamide; (E)-3-(5-(4-chlorophenyl)-1-(2,4-dichlorophenyl)-4-methyl-1H-pyrazole-3-yl)-N-(3-chloropropyl)acrylamide; (E)-3-(5-(4-chlorophenyl)-1-(2,4-dichlorophenyl)-4-methyl-1H-pyrazole-3-yl)-N-hexylacrylamide; (E)-3-(5-(4-chlorophenyl)-1-(2,4-dichlorophenyl)-4-methyl-1H-pyrazole-3-yl)-N-(cyclohexylmethyl)acrylamide; (E)-3-(5-(4-chlorophenyl)-1-(2,4-dichlorophenyl)-4-methyl-1H-pyrazole-3-yl)-N-(3-(trifluoromethyl)benzyl)acrylamide; (E)-3-(5-(4-chlorophenyl)-1-(2,4-dichlorophenyl)-4-methyl-1H-pyrazole-3-yl)-N-(4-(trifluoromethyl)benzyl)acrylamide; (E)-3-(5-(4-chlorophenyl)-1-(2,4-dichlorophenyl)-4-methyl-1H-pyrazole-3-yl)-N-(4-(dimethylamino)benzyl)acrylamide; (E)-N-(3-chlorophenethyl)-3-(5-(4-chlorophenyl)-1-(2,4-dichlorophenyl)-4-methyl-1H-pyrazole-3-yl)acrylamide; (E)-3-(5-(4-chlorophenyl)-1-(2,4-dichlorophenyl)-4-methyl-1H-pyrazole-3-yl)-N-(piperidine-1-yl)acrylamide; (E)-3-(5-(4-chlorophenyl)-1-(2,4-dichlorophenyl)-4-methyl-1H-pyrazole-3-yl)-N-(furan-3-yl methyl)acrylamide; (E)-4-(5-(4-chlorophenyl)-1-(2,4-dichlorophenyl)-4-methyl-1H-pyrazole-3-yl)-1-(piperidine-4-yl)acrylamide; 2-(5-(4-chlorophenyl)-1-(2,4-dichlorophenyl)-4-methyl-1H-pyrazole-3-yl)-2-oxo-N-phenyl acetamide; 2-(5-(4-chlorophenyl)-1-(2,4-dichlorophenyl)-4-methyl-1H-pyrazole-3-yl)-N-methyl-2-oxo-N-phenyl acetamide; N-(3-chlorobenzyl)-2-(5-(4-chlorophenyl)-1-(2,4-dichlorophenyl)-4-methyl-1H-pyrazole-3-yl)-2-oxo-acetamide; N-(3-chlorophenethyl)-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-N-cyclopentyl-2-oxo-acetamide; 2-(5-(4-chlorophenyl)-1-(2,4-dichlorophenyl)-4-methyl-1H-pyrazole-3-yl)-N-cyclohexyl-2-oxo-acetamide; 2-(5-(4-chlorophenyl)-1-(2,4-dichlorophenyl)-4-methyl-1H-pyrazole-3-yl)-N-cycloheptyl-2-oxo-acetamide; 1-(5-(4-chlorophenyl)-1-(2,4-dichlorophenyl)-4-methyl-1H-pyrazole-3-yl)-2-(pyrrolidine-1-yl)ethane-1,2-dione; 1-(5-(4-chlorophenyl)-1-(2,4-dichlorophenyl)-4-methyl-1H-pyrazole-3-yl)-2-(piperidine-1-yl)ethane-1,2-dione; 1-(azepane-1-yl)-2-(5-(4-chlorophenyl)-1-(2,4-dichlorophenyl)-4-methyl-1H-pyrazole-3-yl)ethane-1,2-dione; 2-(5-(4-chlorophenyl)-1-(2,4-dichlorophenyl)-4-methyl-1H-pyrazole-3-yl)-2-oxo-N-(piperidine-1-yl)acetamide; 2-(5-(4-chlorophenyl)-1-(2,4-dichlorophenyl)-4-methyl-1H-pyrazole-3-yl)-N-(cyclohexylmethyl)-2-oxo-acetamide; 1-(5-(4-chlorophenyl)-1-(2,4-dichlorophenyl)-4-methyl-1H-pyrazole-3-yl)-2-(piperazine-1-yl)ethane-1,2-dione; tert-butyl 4-(2-(5-(4-chlorophenyl)-1-(2,4-dichlorophenyl)-4-methyl-1H-pyrazole-3-yl)-2-oxoacetyl)piperazine-1-carboxylate; 1-(5-(4-chlorophenyl)-1-(2,4-dichlorophenyl)-4-methyl-1H-pyrazole-3-yl)-2-(4-methylpiperazine-1-yl)ethane-1,2-dione; 1-(5-(4-chlorophenyl)-1-(2,4-dichlorophenyl)-4-methyl-1H-pyrazole-3-yl)-2-morpholinoethane-1,2-dione; 1-(5-(4-chlorophenyl)-1-(2,4-dichlorophenyl)-4-methyl-1H-pyrazole-3-yl)-2-thiomorpholinoethane-1,2-dione; 2-(5-(4-chlorophenyl)-1-(2,4-dichlorophenyl)-4-methyl-1H-pyrazole-3-yl)-N-adamantyl-2-oxo-acetamide; 2-(5-(4-chlorophenyl)-1-(2,4-dichlorophenyl)-4-methyl-1H-pyrazole-3-yl)-N,N-dicyclohexyl-2-oxo-acetamide; 2-(5-(4-chlorophenyl)-1-(2,4-dichlorophenyl)-4-methyl-1H-pyrazole-3-yl)-N,N-dihexyl-2-oxo-acetamide; 2-(5-(4-chlorophenyl)-1-(2,4-dichlorophenyl)-4-methyl-1H-pyrazole-3-yl)-N-ethyl-2-oxo-acetamide; 2-(5-(4-chlorophenyl)-1-(2,4-dichlorophenyl)-4-methyl-1H-pyrazole-3-yl)-2-oxo-N-propyl acetamide; 2-(5-(4-chlorophenyl)-1-(2,4-dichlorophenyl)-4-methyl-1H-pyrazole-3-yl)-N-isopropyl-2-oxo-acetamide; N-butyl-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-N-isobutyl-2-oxo-acetamide; N-tert-butyl-2-(5-(4-chlorophenyl)-1-(2,4-dichlorophenyl)-4-methyl-1H-pyrazole-3-yl)-2-oxo-acetamide; 2-(5-(4-chlorophenyl)-1-(2,4-dichlorophenyl)-4-methyl-1H-pyrazole-3-yl)-2-oxo-N-pentylacetacetamide; 2-(5-(4-chlorophenyl)-1-(2,4-dichlorophenyl)-4-methyl-1H-pyrazole-3-yl)-N-hexyl-2-oxo-acetamide; 2-(5-(4-chlorophenyl)-1-(2,4-dichlorophenyl)-4-methyl-1H-pyrazole-3-yl)-N-heptyl-2-oxo-acetamide; 2-(5-(4-chlorophenyl)-1-(2,4-dichlorophenyl)-4-methyl-1H-pyrazole-3-yl)-N-octyl-2-oxo-acetamide; and 2-(5-(4-chlorophenyl)-1-(2,4-dichlorophenyl)-4-methyl-1H-pyrazole-3-yl)-N-(2-hydroxyethyl)-2-oxo-acetamide.

4. The method of claim 1, wherein the 1H-pyrazole-3-amide based compound is (E)-N-(3-chlorobenzyl)-3-(5-(4-chlorophenyl)-1-(2,4-dichlorophenyl)-4-methyl-1H-pyrazole-3-yl)acrylamide or 2-(5-(4-chlorophenyl)-1-(2,4-dichlorophenyl)-4-methyl-1H-pyrazole-3-yl)-N-(2-hydroxyethyl)-2-oxo-acetamide.

5. The method of claim 1, wherein the fibrosis is induced in one or more tissues selected from the group consisting of kidney, liver, lung, heart, bone, bone marrow, fat, and skin.
